SEQUENCE LISTING (1 sequence)

SEQ ID NO: 1 - protein, 1087 aa, Homo sapiens
MADHVQSLAQLENLCKQLYETTDTTTRLQAEKALVEFTNSPDCLSKCQLLLERGSSSYSQLLAATCLTKLVSRTNNPLPLEQRIDIRNYVLNYLATRPKLATFVTQALIQLYARITKLGWFDCQKDDYVFRNAITDVTRFLQDSVEYCIIGVTILSQLTNEINQADTTHPLTKHRKIASSFRDSSLFDIFTLSCNLLKQASGKNLNLNDESQHGLLMQLLKLTHNCLNFDFIGTSTDESSDDLCTVQIPTSWRSAFLDSSTLQLFFDLYHSIPPSFSPLVLSCLVQIASVRRSLFNNAERAKFLSHLVDGVKRILENPQSLSDPNNYHEFCRLLARLKSNYQLGELVKVENYPEVIRLIANFTVTSLQHWEFAPNSVHYLLSLWQRLAASVPYVKATEPHMLETYTPEVTKAYITSRLESVHIILRDGLEDPLEDTGLVQQQLDQLSTIGRCEYEKTCALLVQLFDQSAQSYQELLQSASASPMDIAVQEGRLTWLVYIIGAVIGGRVSFASTDEQDAMDGELVCRVLQLMNLTDSRLAQAGNEKLELAMLSFFEQFRKIYIGDQVQKSSKLYRRLSEVLGLNDETMVLSVFIGKIITNLKYWGRCEPITSKTLQLLNDLSIGYSSVRKLVKLSAVQFMLNNHTSEHFSFLGINNQSNLTDMRCRTTFYTALGRLLMVDLGEDEDQYEQFMLPLTAAFEAVAQMFSTNSFNEQEAKRTLVGLVRDLRGIAFAFNAKTSFMMLFEWIYPSYMPILQRAIELWYHDPACTTPVLKLMAELVHNRSQRLQFDVSSPNGILLFRETSKMITMYGNRILTLGEVPKDQVYALKLKGISICFSMLKAALSGSYVNFGVFRLYGDDALDNALQTFIKLLLSIPHSDLLDYPKLSQSYYSLLEVLTQDHMNFIASLEPHVIMYILSSISEGLTALDTMVCTGCCSCLDHIVTYLFKQLSRSTKKRTTPLNQESDRFLHIMQQHPEMIQQMLSTVLNIIIFEDCRNQWSMSRPLLGLILLNEKYFSDLRNSIVNSQPPEKQQAMHLCFENLMEGIERNLLTKNRDRFTQNLSAFRREVNDSMKNSTYGVNSNDMMS

(19) United States
(12) Patent Application Publication
(10) Pub. No.: US 2014/0371158 A1
(43) Pub. Date: Dec. 18, 2014

(54) BEAUVERICIN COMPOSITIONS AND METHODS THEREOF FOR INHIBITING THE HSP90 CHAPERONE PATHWAY

(71) Applicant: Georgia Regents University, Augusta, GA (US)

(72) Inventors: Ahmed Chadli, Evans, GA (US); Abdessamad Debbab, Dusseldorf (DE); Peter Proksch, Dusseldorf (DE)

(21) Appl. No.: 14/305,233

(22) Filed: Jun. 16, 2014

Related U.S. Application Data
(60) Provisional application No. 61/834,966, filed on Jun. 14, 2013.

(51) Int. Cl. A61K 38/15; A61K 45/06

(52) U.S. Cl. CPC A61K 38/15; A61K 45/06
USPC 514/19.4; 514/19.3; 514/19.5; 514/19.6; 435/375

(57) ABSTRACT

Methods of inhibiting the Hsp90 chaperone pathway including contacting one or more target cells with an effective amount of a naturally occurring beauvericin, a synthetic beauvericin, or a derivative, analog or prodrug, or a pharmacologically active salt thereof to reduce, decrease, or inhibit the Hsp90 chaperone pathway in the cells compared to a control are disclosed. The methods can reduce the viability of the target cells, for example, by increasing apoptosis or pro-apoptotic pathways. In preferred embodiments, the methods reduce or do not increase Hsp70, Hsp24, Hsp40, or HOP expression; reduce or do not increase the heat shock response; reduce or do not increase pro-survival pathways in the cells. Methods of treating diseases such as cancer, inflammatory diseases or disorders, neurodegenerative diseases, and infectious diseases using the disclosed compositions and methods are also disclosed.

### CROSS REFERENCE TO RELATED APPLICATIONS

This application claims priority to and benefit of U.S. Provisional Patent Application No. 61/834,966 filed on Jun. 14, 2013, which is incorporated by reference in its entirety.

### STATEMENT REGARDING FEDERALLY SPONSORED RESEARCH OR DEVELOPMENT

This invention was made with Government Support under grant 1R01GM102443-01 awarded to Ahmed Chadli by the National Institutes of Health. The U.S. Government has certain rights in the invention.

### FIELD OF THE INVENTION

The present invention relates generally to the inhibition of the biological activities of the heat shock protein Hsp90 and in particular to the application of beauvericin and its derivatives for inhibition of the Hsp90 pathway.

### BACKGROUND OF THE INVENTION

Heat shock protein 90 (“Hsp90”) is a promising therapeutic target. The inactivation of Hsp90 delivers a combinatorial attack on multiple signaling pathways leading to a more efficient killing of cancer cells and reducing resistance to chemotherapy (Workman, Cancer Letters, 206, 149-157 (2004)). Over 40 clinical trials in phases I-III with 13 small molecule inhibitors of Hsp90 are ongoing worldwide (Neckers, et al., Clinical cancer research, 18, 64-76 (2012)). Most of these inhibitors target the N-terminal ATP binding site to inactivate the ATPase activity of Hsp90, causing proteasomal degradation of its client proteins.

Unfortunately, these N-terminus inhibitors, such as geldanamycin or its analog 17-AAG, also induce overexpression of apoptosis inhibitor proteins Hsp70 and Hsp27. Hsp70 and Hsp27 are thought to be responsible for the modest outcomes in cancer treatments observed in the clinic (Whitesell, et al., Nature Reviews Cancer, 5, 761-772 (2005); Workman, Cancer Letters 206, 149-157 (2004); Neckers, et al., Clinical cancer research, 18, 64-76 (2012); Davenport, et al., Leukemia U.K, 24, 1804-1807 (2010)). Thus, inhibitors of the Hsp90 chaperone with different mechanisms of action are urgently needed.

Therefore, it is an object of the invention to provide new compositions and methods for inhibiting the Hsp90 pathway.

### SUMMARY OF THE INVENTION

Methods and compositions for inhibiting the Hsp90 chaperone pathway are provided. One embodiment provides a method for inhibiting the Hsp90 pathway by contacting one or more target cells expressing Hsp90 with an effective amount of beauvericin, an analog, prodrug, a pharmacologically active salt thereof, or derivative thereof to reduce, decrease, or inhibit the Hsp90 chaperone pathway in the cells compared to a control. The beauvericin can be a naturally occurring beauvericin or a synthetic beauvericin. In some embodiments, the cells have an increased expression of the Hsp90 complex relative to healthy cells, or are under stress or transforming pressure. The disclosed methods can inhibit the formation of Hsp90 complexes. Hsp90 complexes can include one or more co-chaperones.

Other embodiments provide methods that increase the degradation of Hsp90 complexes. The Hsp90 complexes can include one or more co-chaperones or client proteins including, but not limited to AKT, pAKT, CDK4, ILK, Her2, Her3 and the glucocorticoid receptor (GR). Inhibition of the Hsp90 pathway can be achieved by inhibiting Hsp90-mediated folding, activation, or assembly of proteins. The disclosed methods can reduce the viability of the target cells, for example, by increasing apoptosis or pro-apoptotic pathways. In preferred embodiments, the methods reduce or do not increase Hsp70, Hsp24, Hsp40, or HOP expression; reduce or do not increase the heat shock response; reduce or do not increase pro-survival pathways in the cells.

Additional methods can include administering to the subject a second therapeutic agent, for example a chemotherapeutic agent. Methods of treating diseases and conditions such as cancer, inflammatory diseases or disorders, neurodegenerative diseases, and infectious diseases using the disclosed compositions and methods are also disclosed.

### BRIEF DESCRIPTION OF THE DRAWINGS

FIG. 1 is a histogram showing the % hormone binding activity of the progesterone receptor (PR) reconstituted in rabbit reticulocyte lysate without (RL) or with the specified metabolites. Values for hormone binding activity are normalized to the reconstituted PR in the absence of inhibitor (RL; −ve control, 100% chaperone activity). For clarity, beauvericin (AD05) is indicated in the graph by ♦. The representative chemical structure of beauvericin (compound AD05) is illustrated at the top of the graph. “PR” is the PR only. “PR22” is an anti-PR antibody only.

FIG. 2 is a histogram showing the % hormone binding activity of the “A” isoform of the progesterone receptor (PR_{A}) reconstituted in rabbit reticulocyte lysate (RRL) with or without PR_{A} and increasing concentrations of AD05 (beauvericin at 0, 1, 5, 10, 20, 40 μM), respectively. Values for hormone binding activity are normalized to the reconstituted PR in the absence of inhibitor (RRL+PR_{A}+0 μM AD05; −ve control, 100% chaperone activity). “C” is anti-PR_{A} (PR22) antibody only. “PR” is PR_{A} only.

FIG. 3 is an image of a coomassie blue-stained SDS-PAGE gel showing analysis of the protein complexes of FIG. 2. The antibody heavy chain (HC), Hsp90, Hsp70, Hsp40 and Exportin-7 (Exp-7) are indicated by labels to the right of the image. Molecular weight standards are labeled to the left of the image.

FIG. 4 is a histogram showing the % hormone binding activity of the progesterone receptor (PR_{A}) reconstitution using the five purified proteins (Hsp90, Hsp70, Hsp40, HOP and p23) with or without the inhibitors 17-AAG (17AA) and beauvericin (AD05). “C” is PR-specific antibody (PR22), PR is the “A” isoform of progesterone receptor and the antibody of “C.” “5P” is PR with all five purified proteins. Values for hormone binding activity are normalized to the reconstituted PR in the presence of all five proteins (5P; −ve control, 100% chaperone activity).

FIG. 5 is an image of a coomassie blue-stained SDS-PAGE gel showing analysis of the protein complexes of FIG. 4. The positions of the antibody heavy chain (HC), HOP, Hsp90, Hsp70, Hsp40 and PR_{A} are indicated by labels on the right of the image.

FIGS. 6A and 6B are line graphs showing the relative survival of Hs578T (FIG. 6A) and MDA-MB-453 (FIG. 6B) breast cancer cells in the presence of increasing concentrations of AD05 (beauvericin at 0, 2, 4, 6, 8, 10 μM), over time courses of 24 h (♦), 48 h (▪) and 72 h (▴), respectively.

FIGS. 7A and 7B are images of Western blots, showing the cytosol of Hs578T (FIG. 7A) and MAD-MB-453 (FIG. 7B) breast cancer cells treated with 3 μM AD05 (beauvericin) for 48 h and blotted with antibodies against the proteins specified labeled to the right of each image, respectively.

### DETAILED DESCRIPTION OF THE INVENTION

### I. Definitions

The term “Hsp90” includes each member of the family of heat shock proteins having a mass of about 90 kilo Daltons. For example, in humans the highly conserved Hsp90 family includes the cytosolic Hsp90alpha (Hsp90a) and Hsp90beta (Hsp90β) isoforms, as well as GRP94, which is found in the endoplasmic reticulum, and Hsp75/TRAP1, which is found in the mitochondrial matrix.

The term “Hsp90 chaperone pathway or Hsp90 pathway” refers to any process involving the biological activity of Hsp90.

The term “Hsp90 inhibitor” refers to an agent that reduces, decreases, or inhibits the expression or activity of Hsp90 or the Hsp90 chaperone pathway. The agent can directly on Hsp90 or on a protein upstream or downstream of Hsp90 in the Hsp90 pathway.

The terms “AD05” and “beauvericin” refer to the cyclic hexadepsipeptide of Formula I.

The term “effective amount” or “therapeutically effective amount” means a dosage sufficient to provide treatment of the disease state being treated or to otherwise provide a desired pharmacologic and/or physiologic effect. The precise dosage will vary according to a variety of factors such as subject-dependent variables (e.g., age, immune system health, etc.), the disease, and the treatment being effected.

The terms “individual,” “subject,” and “patient” are used interchangeably herein, and refer to a mammal, including, but not limited to, rodents, simians, humans, mammalian farm animals, mammalian sport animals, and mammalian pets.

The terms “treat”, “treatment” and “treating” refer to the reduction or amelioration of the progression, severity and/or duration of a disease or disorder, delay of the onset of a disease or disorder, or the amelioration of one or more symptoms (preferably, one or more discernible symptoms) of a disease or disorder, resulting from the administration of one or more therapies (e.g., one or more therapeutic agents such as a compound of the invention). The terms “treat”, “treatment” and “treating” also encompass the reduction of the risk of developing a disease or disorder, and the delay or inhibition of the recurrence of a disease or disorder.

The terms “reduce”, “inhibit” or “decrease” are used relative to a control. Controls are known in the art. For example a decrease response in a subject or cell treated with a compound is compared to a response in subject or cell that is not treated with the compound.

The term “pharmaceutically acceptable carrier” means one or more carrier ingredients approved by a regulatory agency of the Federal or a state government or listed in the U.S. Pharmacopoeia or other generally recognized pharmacopoeia for use in animals, mammals, and more particularly in humans. Non-limiting examples of pharmaceutically acceptable carriers include liquids, such as water and oils, including those of petroleum, animal, vegetable, or synthetic origin. Water is preferred vehicle when the compound of the invention is administered intravenously. Saline solutions and aqueous dextrose and glycerol solutions can also be employed as liquid vehicles, particularly for injectable solutions.

The term “in combination” refers to the use of more than one therapeutic agent. The use of the term “in combination” does not restrict the order in which said therapeutic agents are administered to a subject.

The term “17-AAG” refers to tanespimycin (17-N-allylamino-17-demethoxygeldanamycin), the derivative of the antibiotic geldanamycin that is a known inhibitor of Hsp90.

“Localization Signal or Sequence or Domain or Ligand” or “Targeting Signal or Sequence or Domain or Ligand” are used interchangeably and refer to a signal that directs a molecule to a specific cell, tissue, organelle, or intracellular region. The signal can be polynucleotide, polypeptide, or carbohydrate moiety or can be an organic or inorganic compound sufficient to direct an attached molecule to a desired location.

### II. Compositions for Inhibiting Hsp90

It has been discovered that the cyclic peptide beauvericin is an inhibitor of the ATP-dependent chaperone Hsp90. It is believed beauvericin inhibits the Hsp90 chaperone pathway by disrupting the Hsp90 machinery. The mechanism is thought to involve nuclear export factor Exportin-7 and is distinct from that of N-terminus inhibitors of Hsp90. For example, beauvericin does not induce overexpression of Hsp70 and Hsp27 and limits the activation of pro-survival pathways associated with N-terminal Hsp90 inhibitors such as 17-AAG.

Pharmaceutical compositions including an effective amount of beauvericin and methods of use thereof for inhibiting the Hsp90 pathway in a subject with enhanced Hsp90 activity relative to a healthy subject are provided. The beauvericin can be a naturally occurring beauvericin, a synthetic beauvericin, or a derivative, analog or prodrug, or a pharmacologically active salt thereof.

A. Heat Shock Protein 90

Heat shock proteins (HSPs) are chaperone proteins that become up-regulated in response to cellular environmental stresses, such as elevated temperature and oxygen or nutrient deprivation. Hsps are chaperones that facilitate the proper folding and repair of other cellular proteins, referred to as “client proteins”, and also aid the refolding of misfolded proteins. Of the several families of Hsps, the Hsp90 family is one of the most abundant, representing approximately 1-2% of the total protein content in non-stressed cells and 4-6% of the protein content of cells that are stressed.

The N-terminal domain of Hsp90 comprises an ATP-binding site that is central to the chaperone function. The C-terminal domain of Hsp90 mediates constitutive Hsp90 dimerization. Conformational changes of Hsp90 are orchestrated with the hydrolysis of ATP.

Hsp90 is highly conserved and facilitates the folding and maturation of over 200 client proteins, which are involved in a broad range of critical cellular pathways and processes. In non-stressed cells Hsp90 participates in low affinity interactions to facilitate protein folding and maturation. In stressed cells Hsp90 can assist the folding of dysregulated proteins, and is known to be involved in the development and maintenance of multiple diseases.

Hsp90 maintains the conformation and stability of many oncogenic proteins, transcription factors, steroid receptors, metalloproteases and nitric oxide synthases that are essential for survival and proliferation of cancer cells (Whitesell, et al., Nature Reviews Cancer, 5, 761-772 (2005)). Thus, Hsp90 client proteins have been associated with the development and progression of cancer. Furthermore, Hsp90 is thought to contribute to maintenance of multiple neurodegenerative diseases that are associated with protein degradation and misfolding (proteinopathy), such as Alzheimer's disease, Huntingdon's disease and Parkinson's disease, through the mis-folding or stabilization of aberrant (neurotoxic) client-proteins.

Inhibition of Hsp90 function results in the misfolding of client proteins, which are subsequently ubiquitinated and degraded through proteasome-dependent pathways. Hence, inactivation of the Hsp90 pathway represents a combinatorial attack on multiple signaling pathways and Hsp90 inhibitors have been developed as therapeutics with efficacy in a broad variety of human diseases.

B. Beauvericin, Derivatives, and Analogs Thereof

1. Beauvericin, Beauvericin A, and Beauvericin B

Beauvericin is a naturally occurring mycotoxin, which has known insecticidal, antimicrobial, antiviral and cytotoxic activities. It is a cyclic hexadepsipeptide that is a trimeric ester of alternating containing D-α-hydroxyisovaleryl and L-N-methylphenylalanyl residues, according to Formula I.

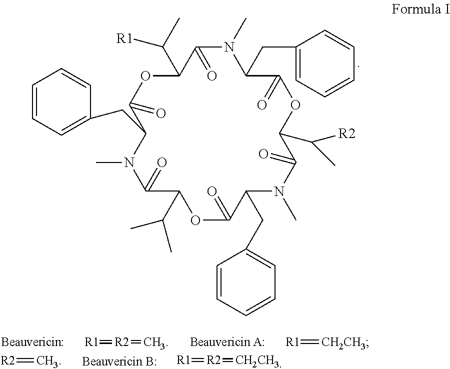

Beauvericin belongs to the enniatin antibiotic family, which is active against gram positive bacteria and mycobacteria. Three different forms of beauvericin are known: a) beauvericin itself, with a molecular weight of 783 daltons and a formula of C₄₅H₅₇N₃O₉, b) beauvericin A, with a molecular weight of 797 daltons and a formula C₄₆H₅₉N₃O₉ and c) beauvericin B with a molecular weight of 811 daltons and a formula C₄₇H₆₁N₃O₉. The presence of one or two additional methyl groups in beauvericin A and B, respectively, gives rise to different lipophilicity amongst these three variants. Beauvericin is incapable of forming intermolecular hydrogen bonds and the absence of any chargeable groups gives rise to poor water solubility and low chemical reactivity.

Beauvericin forms stable associations with metal ions (e.g., Ba²⁺ and Ca²⁺), with an anion-dependent cation specificity. Binding to metal ions results in distinct conformations in beauvericin, which have been associated with membrane-transport functions. It has been proposed that two beauvericin molecules associate either side of a metal cation, which then disassociate at the membrane-water surface, discharging the ion into the water phase. The biological activities of beauvericin are, therefore, likely associated with its ability to unbalance the cellular concentration of cations, especially CA²⁺ (Logrieco, et al. Advances in Microbial Toxin Research and its Biotechnological Exploitation. Upadhyay R. (ed.): pp 23-30 (2002)).

2. Sources of Beauvericin

a. Fungal Fermentation

Beauvericin is a naturally occurring product that can be isolated from endophytic fungi of medicinal plants, such as Aristolochia paucinervis. Fungal genera that are known to produce beauvericin include Beauveria, Paecilomyces, Polyporus, Isaria and Fusarium. Certain Fusarium species, such as Fusarium proliferatum, F. semitectum, F. subglutinans and F. begoniae as well as Beaveria species, such as B. bassiana have been exploited for the commercial production of beauvericin.

Fungal fermentation techniques have been widely used as a means for the large-scale isolation of beauvericin. Mycelial fermentation of fungi, such as Fusarium spp., is a feasible and promising process for the production of beauvericin. Optimization of fungal fermentation processes has increased production of beauvericin to 400 mg/L in the mycelial liquid culture.

b. Synthetic Sources

Beauvericin is commercially available (Sigma-Aldrich product ID B7510) as a powder that is soluble in acetonitrile (1 mg/ml) or methanol (1 mg/ml).

Beauvericin biosynthesis is catalyzed by the 250 kDa multifunctional enzyme beauvericin synthetase, which catalyzes depsipetide formation via a non-ribosomal, thiol-templated mechanism. The enzymatic formation of beauvericin in vitro has been demonstrated using cell-free extracts from the fungus Beauveria bassiana. In analogy to the enniatin synthetase system, formation of beauvericin is strictly dependent on the presence of the constituent amino and hydroxy acid, S-adenosylmethionine, as well as the availability of ATP and Mg²⁺. Synthesizing activity is enriched about 12-fold by fractional ammonium sulfate precipitation (Peeters, et al. J Antibiotics, 13:1762-1766 (1986)).

C. Analogs of Beauvericin

In some embodiments, the beauvericin is an analog of beauvericin.

Analogs of beauvericin are known in the art. See, for example, Matthes, et al., Chem. Commun., 48:5674-5676 (2012) and its supporting, which are specifically incorporated by reference herein in their entirety. Matthes describes analogs of beauvericins synthesized using the non-ribosomal peptide synthetase BbBEAS from the entomopathogenic fungus Beauveria bassiana. Chemical diversity was generated by in vitro chemoenzymatic and in vivo whole cell biocatalytic syntheses using either a B. bassiana mutant or an E. coli strain expressing the bbBeas gene.

D. Formulations

The disclosed compositions containing beauvericin or a derivative, analog or prodrug, or a pharmacologically active salt thereof for the inhibition of Hsp90 can be formulated as pharmaceutical compositions.

Pharmaceutical compositions may be for administration by oral, parenteral (intramuscular, intraperitoneal, intravenous (IV) or subcutaneous injection), transdermal (either passively or using iontophoresis or electroporation), transmucosal (nasal, vaginal, rectal, or sublingual) routes of administration or using bioerodible inserts and can be formulated in unit dosage forms appropriate for each route of administration.

1. Parenteral Administration

In one embodiment, the compositions are administered in an aqueous solution, by parenteral injection. The formulation may also be in the form of a suspension or emulsion. In general, pharmaceutical compositions are provided including effective amounts of beauvericin, or a derivative, analog or prodrug, or a pharmacologically active salt thereof and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN®20, TWEEN®80, Polysorbate 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

2. Enteral Administration

The compositions can be formulated for oral delivery.

a. Additives for Oral Administration

Oral solid dosage forms are described generally in Remington's Pharmaceutical Sciences, 18th Ed. 1990 (Mack Publishing Co. Easton Pa. 18042) at Chapter 89. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions may influence the physical state, stability, rate of in vivo release, and rate of in vivo clearance of the present proteins and derivatives. See, e.g., Remington's Pharmaceutical Sciences, 18th Ed. (1990), Mack Publishing Co., Easton, Pa. 18042) pages 1435-1712 which are herein incorporated by reference. The compositions may be prepared in liquid form, or may be in dried powder (e.g., lyophilized) form. Liposomal or proteinoid encapsulation may be used to formulate the compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). See also Marshall, K. In: Modern Pharmaceutics Edited by G. S. Banker and C. T. Rhodes, Chapter 10, 1979. In general, the formulation will include the peptide (or chemically modified forms thereof) and inert ingredients which protect peptide in the stomach environment, and release of the biologically active material in the intestine.

Another embodiment provides liquid dosage forms for oral administration, including pharmaceutically acceptable emulsions, solutions, suspensions, and syrups, which may contain other components including inert diluents; adjuvants such as wetting agents, emulsifying and suspending agents; and sweetening, flavoring, and perfuming agents.

Controlled release oral formulations may be desirable. Beauvericin, or a derivative, analog or prodrug, or a pharmacologically active salt thereof can be incorporated into an inert matrix which permits release by either diffusion or leaching mechanisms, e.g., gums. Slowly degenerating matrices may also be incorporated into the formulation. Another form of a controlled release is based on the Oros therapeutic system (Alza Corp.), i.e., the drug is enclosed in a semipermeable membrane which allows water to enter and push drug out through a single small opening due to osmotic effects. For oral formulations, the location of release may be the stomach, the small intestine (the duodenum, the jejunum, or the ileum), or the large intestine. Preferably, the release will avoid the deleterious effects of the stomach environment, either by protection of the peptide (or derivative) or by release of the peptide (or derivative) beyond the stomach environment, such as in the intestine. To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

b. Chemically Modified Forms for Oral Dosage

Beauvericin, or a derivative, analog or prodrug, thereof may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. PEGylation is a preferred chemical modification for pharmaceutical usage. Other moieties that may be used include: propylene glycol, copolymers of ethylene glycol and propylene glycol, carboxymethyl cellulose, dextran, polyvinyl alcohol, polyvinyl pyrrolidone, polyproline, poly-1,3-dioxolane and poly-1,3,6-tioxocane (see, e.g., Abuchowski and Davis (1981) “Soluble Polymer-Enzyme Adducts,” in Enzymes as Drugs. Hocenberg and Roberts, eds. (Wiley-Interscience: New York, N.Y.) pp. 367-383; and Newmark, et al. (1982) J. Appl. Biochem. 4:185-189).

3. Controlled Delivery Polymeric Matrices

Controlled release polymeric devices can be made for long term release systemically following implantation of a polymeric device (rod, cylinder, film, disk) or injection (microparticles). The matrix can be in the form of microparticles such as microspheres, where peptides are dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of beauvericin, although biodegradable matrices are preferred. These may be natural or synthetic polymers, although synthetic polymers are preferred due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be cross-linked with multivalent ions or polymers.

The matrices can be formed by solvent evaporation; spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5, 13-22 (1987); Mathiowitz, et al., Reactive Polymers, 6, 275-283 (1987); and Mathiowitz, et al., J. Appl. Polymer Sci., 35, 755-774 (1988).

The devices can be formulated for local release to treat the area that is subject to a disease, which will typically deliver a dosage that is much less than the dosage for treatment of an entire body or systemic delivery. These can be implanted or injected subcutaneously, into the muscle, fat, or swallowed.

4. Topical Administration

Topical administration of beauvericins may be desirable. In some embodiments beauvericin, or a derivative, analog or prodrug, or a pharmacologically active salt thereof can be incorporated into an inert matrix to be administered in the form of a suppository or pessary, or may be applied topically in the form of a lotion, solution, cream, ointment or dusting powder. Beauvericins may also be transdermally administered, for example, by the use of a skin patch or other intra-dermal devices. They may also be administered by the ocular route. For application topically to the skin, the beauvericins can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

In some embodiments, the topical administration is in the mouth. Formulations suitable for topical administration in the mouth include lozenges comprising the beauvericin in a flavored basis, usually sucrose and acacia or tragacanth; pastilles comprising the beauvericin in an inert basis such as gelatin and glycerin, or sucrose and acacia; and mouthwashes comprising the beauvericin in a suitable liquid carrier.

E. Targeting Moieties

In some embodiments, the composition includes a targeting signal, a protein transduction domain or a combination thereof. The targeting moiety can be attached or linked directly or indirectly to beauvericin, or a derivative, analog or prodrug thereof. For example, in preferred embodiments, the targeting moiety is attached or linked to a beauvericin delivery vehicle such as a nanoparticle or a microparticle.

The targeting signal or sequence can be specific for a host, tissue, organ, cell, organelle, non-nuclear organelle, or cellular compartment. Moreover, the compositions disclosed here can be targeted to other specific intercellular regions, compartments, or cell types.

In one embodiment, the targeting signal binds to its ligand or receptor which is located on the surface of a target cell such as to bring the beauvericin and cell membranes sufficiently close to each other to allow penetration of the beauvericin into the cell. Additional embodiments of the present disclosure are directed to specifically delivering beauvericin, or a derivative, analog or prodrug, or a pharmacologically active salt thereof to specific tissue or cell types with undesirable Hsp90 activity. In a preferred embodiment, the targeting molecule is selected from the group consisting of an antibody or antigen binding fragment thereof, an antibody domain, an antigen, a T-cell receptor, a cell surface receptor, a cell surface adhesion molecule, a major histocompatibility locus protein, a viral envelope protein and a peptide selected by phage display that binds specifically to a defined cell.

Beauvericin can be attached to polymeric particles directly or indirectly though adaptor elements which interact with the polymeric particle. The polymeric particles can microparticles or nanoparticles. Adaptor elements may be attached to polymeric particles in at least two ways. The first is during the preparation of micro- and nanoparticles, for example, by incorporation of stabilizers with functional chemical groups during emulsion preparation of microparticles. For example, adaptor elements, such as fatty acids, hydrophobic or amphiphilic peptides and polypeptides can be inserted into the particles during emulsion preparation. In a second embodiment, adaptor elements may be amphiphilic molecules such as fatty acids or lipids which may be passively adsorbed and adhered to the particle surface, thereby introducing functional end groups for tethering to ligands. Adaptor elements may associate with micro and nanoparticles through a variety of interactions including, but not limited to, hydrophobic interactions, electrostatic interactions and covalent coupling.

Exemplary targeting signals include a binding moiety such as an antibody or antigen binding fragment thereof specific for a receptor expressed at the surface of a target cell or other specific antigens, such as cancer antigens. Representative receptors include but are not limited to growth factors receptors, such as epidermal growth factor receptor (EGFR; HER1; c-erbB2 (HER2); c-erbB3 (HER3); c-erbB4 (HER4); insulin receptor; insulin-like growth factor receptor 1 (IGF-1R); insulin-like growth factor receptor 2/Mannose-6-phosphate receptor (IGF-II R/M-6-P receptor); insulin receptor related kinase (IRRK); platelet-derived growth factor receptor (PDGFR); colony-stimulating factor-1receptor (CSF-1R) (c-Fms); steel receptor (c-Kit); Flk2/Flt3; fibroblast growth factor receptor 1 (Flg/Cek1); fibroblast growth factor receptor 2 (Bek/Cek3/K-Sam); Fibroblast growth factor receptor 3; Fibroblast growth factor receptor 4; nerve growth factor receptor (NGFR) (TrkA); BDNF receptor (TrkB); NT-3-receptor (TrkC); vascular endothelial growth factor receptor 1 (Flt1); vascular endothelial growth factor receptor 2/Flk1/KDR; hepatocyte growth factor receptor (HGF-R/Met); Eph; Eck; Eek; Cek4/Mek4/HEK; Cek5; Elk/Cek6; Cek7; Sek/Cek8; Cek9; Cek10; HEK11; 9 Ror1; Ror2; Ret; Axa; RYK; DDR; and Tie.

In some embodiments, the targeting signal is or includes a protein transduction domain, also known as cell penetrating peptides (CPPS). PTDs are known in the art, and include but are not limited to small regions of proteins that are able to cross a cell membrane in a receptor-independent mechanism (Kabouridis, P., Trends in Biotechnology, (11):498-503 (2003)). The two most commonly employed PTDs are derived from TAT (Frankel and Pabo, Cell, December 23; 55(6):1189-93 (1988)) protein of HIV and Antennapedia transcription factor from Drosophila, whose PTD is known as Penetratin (Derossi et al., J Biol Chem., 269(14):10444-50 (1994)).

### III. Methods of Treatment

Beauvericin can be used to treat a variety of diseases and disorders including, but not limited to cancer. The Hsp90 chaperone complex assists in the folding and function of a variety of disease-associated ‘client proteins’. Multiple disease-associated proteins, for example those involved in cell-specific oncogenic processes and neurodegenerative disorders, have been shown to be regulated or protected by the binding of the Hsp90 machinery. One embodiment provides a method for inhibit the Hsp90 pathway by administering to a subject in need thereof an effective amount of beauvericin, a prodrug, derivative, or analog thereof to inhibit the Hsp90 pathway in cells having undesirable Hsp pathway expression without an increase in expression of Hsp70 and/or Hsp27. In the context of cancer, these can include BCR-ABL in the chronic myelogenous leukemia (CML), nucleophosmin-anaplastic lymphoma kinase (NPM-ALK) in lymphomas, mutated FLT3 in acute myeloid leukemia, EGFR harboring kinase mutations in non-small cell lung cancer (NSCLC), the zeta-associated protein of 70 kDa (ZAP-70) as expressed in patients with aggressive chronic lymphocytic leukemia (CLL), mutant B-Raf in melanoma, human epidermal growth factor receptor 2 (HER2) in HER2-overexpressing breast cancer, mutant c-Kit in gastrointestinal stromal tumors (GIST), and activated Akt in small cell lung carcinoma, to list a few.

It is now accepted that at the phenotypic level, the Hsp90 machinery serves as a biochemical buffer for the numerous cancer-specific lesions that are characteristic of diverse tumors and the successful validation of Hsp90 as a target in cancer through the use of pharmacologic agents led to the development of a number of Hsp90 inhibitors which have been the subject of numerous clinical trials (reviewed in Taldone, et al., Curr. Opin. Pharmacol., 8(4): 370-374 (2008)). Although a number of Hsp90 inhibitors are known in the art, most known Hsp90 inhibitors work by binding to the N-terminus of Hsp90 act by binding to the N-terminus of Hsp90 and disrupting the interaction between Hsp90 and heat shock factor 1 (HSF-1). Such Hsp90 inhibitors also induce an increase in expression of Hsp70 and/or Hsp27, which are associated with pro-survival pathways (Bagatell, et al., Clin Cancer Res., 6(8):3312-8 (2000)). For example, overexpression of Hsp70 has been shown to be indicative of metastasis and poor prognosis in breast cancer patients and to correlate with drug and chemotherapy resistance (Ciocca, et al., J. Natl. Cancer Inst., 85(7):570-4 (1993) and Barnes, et al., Cell Stress Chaperones, 6(4):316-25. (2001)).

A. Methods of Using Beauvericins

Methods of inhibiting the Hsp90 chaperone pathway are disclosed. The methods typically include contacting one or more cells with an effective amount of a naturally occurring beauvericin, a synthetic beauvericin, or a derivative, analog or prodrug, or a pharmacologically active salt thereof to reduce, decrease, or inhibit the Hsp90 chaperone pathway in the cells compared to a control. The cells are typically characterized as over-expressing one or more Hsp90 complex component, for example Hsp90, or under stress, or under transforming pressure, relative to a control. The cells can be diseased cells.

The methods of use disclosed herein typically include contacting cells with an effective amount of beauvericin, a synthetic beauvericin, or a derivative, analog or prodrug, or a pharmacologically active salt thereof. The contacting can occur in vitro or in vivo. In preferred embodiments, beauvericin, or a derivative, analog or prodrug, or a pharmacologically active salt thereof is administered to a subject in need thereof. The subject can have a disease or disorder caused or exacerbated by proteins protected by the Hsp90 chaperone pathway.

1. Effective Amounts

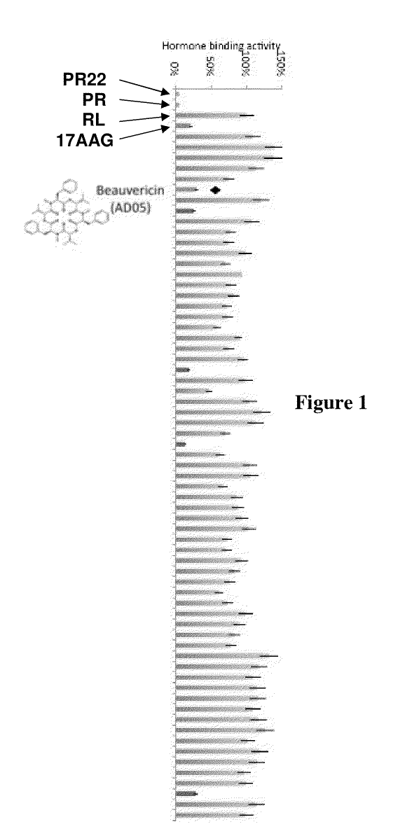

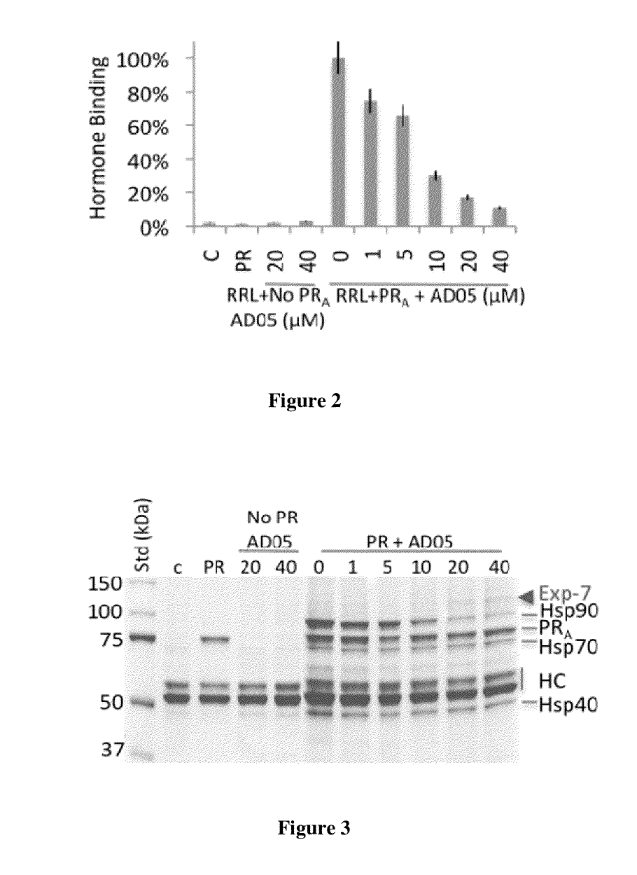

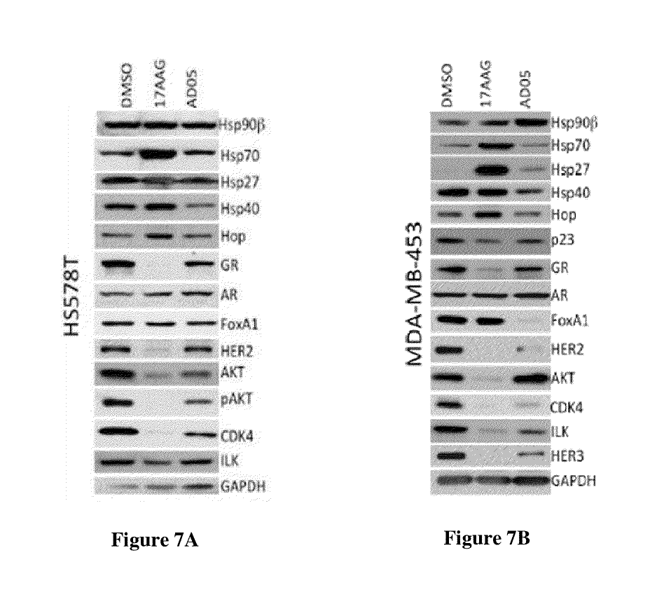

As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The amount of beauvericin contacted with the cells is typically enough to reduce, decrease, or inhibit the Hsp90 chaperone pathway in cells. The Examples below illustrate that beauvericin inhibited the reconstitution of heat-denatured protein by chaperones in a concentration-dependent manner (FIGS. 1 and 2) and caused cellular degradation of several kinase protein clients of Hsp90 (AKT, pAKT and CDK4, ILK, Her2 and Her3) and the glucocorticoid receptor (GR) (FIGS. 7A-B). Therefore, in some embodiments beauvericin, or a derivative, analog or prodrug salt thereof can reduce or inhibit Hsp90-mediated folding, activation, assembly, or function of denatured proteins; increase the degradation of Hsp90 complexes including co-chaperones or client proteins; reduce or inhibit direct association of Hsp90 with death proteins; or a combination thereof. In some embodiments, beauvericin, or a derivative, analog or prodrug thereof increases apoptosis, reduces proliferation, or a combination thereof.

In preferred embodiments, beauvericin, or a derivative, analog or prodrug, thereof does not induce or increase expression of pro-survival pathways. As illustrated in the Examples below, in contrast to the N-terminal inhibitor 17-AAG, beauvericin had no inhibitory activity on the five well-characterized chaperone proteins Hsp90, Hsp70, Hsp40, HOP per se and did not induce overexpression of Hsp70 and Hsp27. As discussed above, over-expression of the apoptosis inhibitor proteins Hsp70 and Hsp27 is associated some Hsp90 inhibitors including 17-AAG and is thought to reduce efficacy of the inhibitors in some uses of Hsp90 inhibitors, for example, the treatment of cancer. Together these data indicate that beauvericin inhibits the chaperone activity of Hsp90 through a mechanism that is distinct from that of 17-AAG and does not induce cellular heat shock response.

Therefore, in some embodiments, beauvericin, or a derivative, analog or prodrug thereof does not reduce or inhibit one or more of the chaperone proteins Hsp90, Hsp70, Hsp40, or HOP itself. For example, the beauvericin can reduce or inhibit the activity of the Hsp90 chaperone complex as a whole without directly inhibiting Hsp90 itself.

In preferred embodiments, beauvericin, or a derivative, analog or prodrug thereof does not induce or increase expression of Hsp70, Hsp27, Hsp40, or HOP or a combination thereof. In some embodiments, beauvericin, or a derivative, analog or prodrug thereof reduces or decreases expression of Hsp70, Hsp27, Hsp40, or HOP or a combination thereof. In some embodiments, beauvericin, or a derivative, analog or prodrug thereof does not increase or induce cellular heat shock response. In some embodiments, beauvericin increases the expression, bioactivity, localization, or the incorporation into client protein complexes of the nuclear export factor Exportin-7, which in turn interferes with connections between Hsp90 and the nucleocytoplasmic trafficking machinery.

2. Controls

The effect of a beauvericin can be compared to control. For example, in some embodiments, one or more of the pharmacological or physiological markers or pathways affected by beauvericin treatment is compared to the same pharmacological or physiological marker or pathway in untreated control cells or untreated control subjects. In preferred embodiments the cells or the subject suffers the same disease or conditions as the treated cells or subject. For example, beauvericin treated cells or subjects can be compared to cells or subjects treated with other Hsp90 inhibitors, such as 17-AAG. The cells or subjects treated with other Hsp90 inhibitors can have a greater increase in Hsp70 expression, Hsp27 expression, or a greater increase in pro-survival signaling than do cells or subjects treated with beauvericin, or a derivative, analog or prodrug thereof.

In preferred embodiments, beauvericin, or a derivative, analog or prodrug thereof is effective to reduce, inhibit, or delay one or more symptoms of a disease in a subject. Diseases that can be treated using the disclosed compositions are discussed in more detail below.

Beauvericin, or a derivative, analog or prodrug, or a pharmacologically active salt thereof can be administered enterally or parenterally. Beauvericin, or a derivative, analog or prodrug, or a pharmacologically active salt thereof can be part of a pharmaceutical composition that includes a pharmaceutically acceptable carrier.

B. Therapeutic Administration

Pharmaceutical compositions including beauvericin, or a derivative, analog or prodrug, or a pharmacologically active salt thereof, may be administered in a number of ways depending on whether local or systemic treatment is desired, and depending on the area to be treated. For example, the disclosed compositions can be administered intravenously, intraperitoneally, intramuscularly, subcutaneously, intracavity, or transdermally. The compositions may be administered orally, parenterally (e.g., intravenously), by intramuscular injection, by intraperitoneal injection, transdermally, extracorporeally, ophthalmically, vaginally, rectally, intranasally, topically or the like, including topical intranasal administration or administration by inhalant. Parenteral administration of the composition, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained.

For all of the disclosed compounds, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. Generally dosage levels of 0.001 to 100 mg/kg of body weight daily are administered to mammals. Generally, for intravenous injection or infusion, dosage may be lower. Preferably, the compositions are formulated to achieve a beauvericin serum level of about 1 to about 1000 μM.

C. Diseases to be Treated

The compositions and methods disclosed herein can be used to treat a variety of diseases and disorders in which blockade of the Hsp90 chaperone pathway is desirable. Hsp90 is a molecular chaperone with important roles in maintaining the functional stability and viability of cells under a transforming pressure. Accordingly, if the Hsp90 is stabilizing diseased or pathogenic cells, it can be desirable to inhibit the Hsp90 chaperone pathway and thereby reduce the viability of the diseased or pathogenic cells.

Therefore, the compositions and methods disclosed herein can be used to treat any disease or disorder in which the Hsp90 chaperone pathway stabilizes or refolds proteins that play a pathogenic role in the diseases or disorder. In some embodiments, Hsp90 or a complex thereof is increased in the cells that express the proteins. Exemplary diseases are provided below.

Beauvericin, or a derivative, analog or prodrug, or a pharmacologically active salt thereof can be administered in an effective amount to increase apoptosis of a cell type or cell types, or a subpopulation of cells that are being protected by the Hsp90 pathway, for example, cells that are expressing pathogenic proteins. In some embodiments, the target cell type or types are under stress or a transforming pressure.

1. Cancer

The compositions and method can be used to treat cancer. Cytotoxic activity of beauvericin has been observed in multiple non-human and human cancer cell lines, including monocytic lymphoma cells (Calo, et al., Pharmacol Res, 49, 73-77 (2004)), breast MCF-7 cells (Zhan, et al., J Nat Prod, 70, 227-232 (2007)), glioma SF-268 cells (Zhan, et al., J Nat Prod, 70, 227-232 (2007)), leukemia CCRF-CEM cells (Jow, et al., Cancer Lett, 216, 165-173 (2004)), non-small cell lung cancer A549 (Lin, et al., Cancer Lett, 230, 248-259 (2005)) and NCI-H460 cells (Zhan, et al., J Nat Prod, 70, 227-232 (2007)), pancreatic carcinoma MIA Pa Ca-2 cells (Zhan, et al., J Nat Prod, 70, 227-232 (2007)), human promyelocytic leukemia HL-60 cells and African green monkey kidney fibroblast cells. Therefore, cancers which can be treated using the compositions and methods described herein include sarcomas, lymphomas, leukemias, carcinomas, blastomas, and germ cell tumors.

A representative but non-limiting list of cancers that the disclosed compositions and methods can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

The examples below show that beauvericin inhibited Hsp90 and induced apoptosis in both the Hs578T and MDA-MB-453 breast cancer cell lines in vitro (FIGS. 7 and 8). Therefore, in a preferred embodiment, beauvericin is used in method of treating breast cancer or a metastasis or secondary cancer derived therefrom.

2. Inflammatory Diseases and Disorders

It has been demonstrated that Hsp90 inhibitors are able to block the activity of certain proinflammatory mediators in different cell types (Malhotra, et al., Am. J. Respir. Cell Mol. Biol., 25:92-97 (2001) and Wax, et al., Arthritis Rheum., 48:541-550 (2003). Moreover, the Hsp90 inhibitor (17-AAG) is able to attenuate inflammation in several diseases (Dello, et al., J. Neurochem., 99:1351-1362 (2006), Chatterjee, Am. J. Respir. Crit. Care Med., 176:667-675 (2007), and Poulaki, FASEB J., 21:2113-2123 (2007)). Kasperkiewicz, et al., Blood, 117(23):6135-6142 (2011) describes that T cells are targets of anti-Hsp90 treatment in autoimmunity to type VII collagen, and Madrigal-Matute, et al., Cardiovascular Research, 86, 330-337 (2010) describes that heat shock protein 90 inhibitors attenuate inflammatory responses in atherosclerosis. Therefore, in some embodiments, the compositions and methods disclosed herein are used to treat an inflammatory response, or an inflammatory or autoimmune disorder or disorder.

Representative inflammatory responses or autoimmune diseases that can be treated include, but are not limited to, rheumatoid arthritis, systemic lupus erythematosus, alopecia areata, anklosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopenic purpura (ATP), Behchet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis—juvenile, discoid lupus, essential mixed cryoglobulinemia, fibromyalgia-fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

In preferred embodiments, beauvericin, or a derivative, analog or prodrug, or a pharmacologically active salt thereof is used to treat atherosclerosis, acute or chronic inflammation, rheumatoid arthritis, encephalomyelitis, systemic lupus erythematosus, autoimmune blistering skin diseases, and uveitis.

3. Neurodegenerative Diseases

Studies indicate that Hsp90 serves as a biochemical buffer for the numerous aberrant processes that facilitate the evolution of the neurodegenerative disease and inhibition of Hsp90 by Hsp90 inhibitors, such as beauvericin, can result in the destabilization of the Hsp90/aberrant protein complexes leading to degradation of these proteins by a proteasome-mediated pathway (Lou, et al., Molecular Neurodegeneration, 5:24 (2010)). Therefore, the compositions and methods disclosed herein can be used to treat neurodegenerative diseases and other proteinopathies and amyloidoses.

a. Proteinopathies

Methods of treating or preventing proteinopathies and amyloidosis are disclosed. For example, the methods can include administering to a subject in need thereof an effective amount of one or more of the disclosed compositions to reduce, delay, or inhibit the expression or accumulation of one or more misfolded proteins.

The compositions can be administrated to a subject in an effective amount to treat a proteinopathy, or symptom, characteristic or comorbidity thereof. Proteinopathies include, but are not limited to, Alzheimer's disease, cerebral β-amyloid angiopathy, Retinal ganglion cell degeneration in glaucoma, Prion diseases, Parkinson's disease and other synucleinopathies, Tauopathies, Frontotemporal lobar degeneration (FTLD), FTLD-FUS, Amyotrophic lateral sclerosis (ALS), Huntington's disease and other triplet repeat disorders, Familial British dementia, Familial Danish dementia, Hereditary cerebral hemorrhage with amyloidosis (Icelandic) (HCHWA-I), CADASIL, Alexander disease, Seipinopathies, Familial amyloidotic neuropathy, Senile systemic amyloidosis, Serpinopathies, AL (light chain) amyloidosis (primary systemic amyloidosis), AH (heavy chain) amyloidosis, AA (secondary) amyloidosis, Type II diabetes, Aortic medial amyloidosis, ApoAl amyloidosis, ApoAll amyloidosis, ApoAIV amyloidosis, Familial amyloidosis of the Finnish type (FAF), Lysozyme amyloidosis, Fibrinogen amyloidosis, Dialysis amyloidosis, Inclusion body myositis/myopathy, Cataract, Retinitis pigmentosa with rhodopsin mutations, Medullary thyroid carcinoma, Cardiac atrial amyloidosis, Pituitary prolactinoma, Hereditary lattice corneal dystrophy, Cutaneous lichen amyloidosis, Mallory bodies, Corneal lactoferrin amyloidosis, Pulmonary alveolar proteinosis, Odontogenic (Pindborg) tumor amyloid, Seminal vesicle amyloid, Cystic Fibrosis, Sickle cell disease, and Critical illness myopathy (CIM).

In certain embodiments the subject has a mutation in a gene, such as the AP, ABri, ADan, superoxide dismutase, a-synuclein, huntingtin, ataxins, or neuroserpin genes, which could lead to accumulation of malformed protein or protein aggregates which could trigger a pathological cascade leading to clinical manifestation of a proteinopathy. The subject may or may not be exhibiting physical symptoms of the proteinopathy at the time treatment is initiated.

b. Amyloidosis

The compositions can also be administrated to a subject in an effective amount to treat amyloidosis, or symptom, characteristic or comorbidity thereof. In some embodiments, the amyloidosis is caused by the amyloid protein beta amyloid (Aβ), medin (AMed), Apolipoprotein AI (AApoA1), atrial natriuretic factor (AANF), Cystatin (ACys), IAPP (Amylin) (AIAPP), beta 2 microglobulin (Aβ2M), Transthyretin (ATTR), Gelsolin (AGel), Lysozyme (ALys), huntingtin, keratoepithelin (Aker), calcitonin (ACal), alpha-synuclein, Prolactin (APro), serum amyloid A, (AA), S-IBM, immunoglobulin light chain AL (AL), or PrPSc (APrP).

Amyloidosis includes, but is not limited to, diseases such as Alzheimer's disease (beta amyloid), aortic medial amyloid (Medin), atherosclerosis (Apolipoprotein AI), cardiac arrhythmias and isolated atrial amyloidosis (atrial natriuretic factor), cerebral amyloid angiopathy (beta amyloid), cerebral amyloid angiopathy—Icelandic type (Cystatin), diabetes mellitus type 2 (IAPP-Amylin), dialysis related amyloidosis (beta 2 microglobulin), familial amyloid polyneuropathy, (transthyretin), finnish amyloidosis (gelsolin), hereditary non-neuropathic systemic amyloidosis (lysozyme), Huntington's disease, (Huntingtin), lattice corneal dystrophy (keratoepithelin), medullary carcinoma of the thyroid (calcitonin), multiple myeloma (paraprotein), Parkinson's disease (alpha-synuclein) prolactinomas (prolactin), rheumatoid arthritis (serum amyloid A), Sporadic Inclusion Body Myositis (S-IBM); systemic AL amyloidosis (immunoglobulin light chain AL), primary cutaneous amyloidosis, AA amyloidosis, senile amyloid of atria of heart, familial visceral amyloidosis, Cerebral amyloid angiopathy (British-type and Danish-type), medullary carcinoma of the thyroid, familial corneal amyloidosis, prion disease systemic amyloidosis, leptomeningeal amyloidosis, haemodialysis-associated amyloidosis, and transmissible spongiform encephalopathies (PrPSc).

Examples of transmissible spongiform encephalopathies include, but are not limited to, human diseases such as Creutzfeld Jakob Disease, variant Creutzfeld Jakob Disease, Gerstmann-Sträussler-Scheinker syndrome (GSS), fatal familial insomnia (FFI), kuru and Alpers' syndrome, and non-human diseases such as bovine spongiform encephalopathy (BSE, commonly known as mad cow disease) in cattle, chronic wasting disease (CWD) in elk and deer, and scrapie in sheep, transmissible mink encephalopathy, feline spongiform encephalopathy and ungulate spongiform encephalopathy.

c. Tauopathies

The disclosed compositions and methods can also be used to treat diseases characterized by increased tau expression, increased tau phosphorylation, or pathologies associated with the aggregation of tau protein in the brain.

Examples of tauopathies and conditions associated therewith include, but are not limited to Alzheimer's disease, Argyrophilic grain disease (AGD), Chronic Traumatic Encephalopathy (CTE), dementia pugilistica (chronic traumatic encephalopathy), frontotemporal dementia, frontotemporal lobar degeneration, ganglioglioma, gangliocytoma, Lytico-Bodig disease (Parkinson-dementia complex of Guam), meningioangiomatosis, Frontotemporal Dementia and Parkinsonism linked to chromosome 17 (FTDP-17), Pick's disease, progressive supranuclear palsy, subacute sclerosing panencephalitis, Tangle-predominant dementia, lead encephalopathy, tuberous sclerosis, Hallervorden-Spatz disease, lipofuscinosis, corticobasal degeneration.

4. Infectious Diseases

The compositions and methods can be used to treat infectious diseases.

a. Viral Infections

Hsp90 is used by numerous DNA and RNA viruses to mediate the activity and maturation of various viral proteins (reviewed in Geller, et al., Biochimica et biophysica acta, 1823:698-706 (2012) and Xiao, et al., Archives of Virology, 155:1021-1031 (2010)). Therefore, Hsp90 inhibitors display broad-spectrum antiviral activity. Although many antiviral drugs eventually produce drug-resistant viral variants that escape inhibition, drug-resistance did not emerge when Hsp90 inhibitors were used to block poliovirus replication, indicating that these types of inhibitors may be refractory to the development of drug resistance (Geller, Genes & Development, 21: 195-205 (2007)). The broad-spectrum antiviral activity of Hsp90 inhibitors and their low propensity for eliciting drug resistance make Hsp90 inhibitors attractive candidates for antiviral therapy (Geller, et al., PLoS ONE, 8(2):e56762 (2013)).

Therefore, in some embodiments, the compositions and methods disclosed herein are used to treat a viral infection. Exemplary viruses include, but are not limited to, Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenzavirus A and B and C), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxviridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae.

b. Fungal Infections

Hsp90 also enables the emergence and maintenance of drug resistance in diverse fungal species (Cowen, et al., Eukaryot Cell, 5:2184-2188 (2006) and Cowen, et al., Science, 309:2185-2189 (2005)). For the most prevalent fungal pathogen of humans, C. albicans, Hsp90 mediates resistance to the azoles, which inhibit ergosterol biosynthesis and are the most widely used class of antifungals in the clinic. Pharmacological inhibition of Hsp90 blocks the emergence of azole resistance and abrogates resistance of laboratory mutants and strains that evolved resistance in a human host. Therefore, in some embodiments, the compositions and methods disclosed herein are used to treat a fungal-related disease or disorder. The compositions can be used to create or rescue drug sensitivity. Therefore, in some embodiments, beauvericin, or a derivative, analog or prodrug, or a pharmacologically active salt thereof is used in combination with a second active agent that is an anti-fungal. Exemplary anti-fungal agents and fungi that can be treated are discussed in Cowen, et al., 106(8):2818-2823 (2009) which is specifically incorporated by reference herein in its entirety.

5. Endothelial Barrier Functions and Lung Inflammation

Hsp90 inhibitors protect the endothelial barrier from dysfunction induced by several inflammatory mediators that are involved in pathogenesis of ALI, ARDS, and other pulmonary inflammatory diseases (Antonov, et al., Am. J. Respir. Cell Mol. Biol., 39:551-559 (2008)). Therefore, the compositions disclosed herein can be used a therapeutic agent in the regulation of endothelial barrier function and lung inflammation.

D. Combination Therapies

The compositions of beauvericin disclosed herein can be used in combination with one or more additional therapeutic agents. The term “combination” or “combined” is used to refer to either concomitant, simultaneous, or sequential administration of two or more agents. Therefore, the combinations can be administered either concomitantly (e.g., as an admixture), separately but simultaneously (e.g., via separate intravenous lines into the same subject), or sequentially (e.g., one of the compounds or agents is given first followed by the second). The additional therapeutic agents can be administered locally or systemically to the subject, or coated or incorporated onto, or into a device.

The additional agent or agents can modulate the Hsp90 chaperone pathway, or beauvericin itself. For example, the additional agent can enhance or reduce the activity of the Hsp90 chaperone pathway, or beauvericin itself. The additional agent or agents can be a second therapeutic that is used to enhance the therapeutic effect of beauvericin, or a derivative, analog or prodrug, or a pharmacologically active salt thereof by targeting a second molecular pathway relevant to the disease, disorder, or condition being treated. In some embodiments, the one or more additional agent is a conventional therapeutic agent for the disease, disorder, or condition to be treated. For example, if the disease to be treated is cancer, a conventional therapeutic agent can be chemotherapy.

It is believed that Hsp90 inhibitors can be used to increase the sensitivity of target cells to some conventional therapeutic agents. Therefore, in some embodiments, the second (conventional) therapeutic agent is used at a lower dosage or for a shorter duration than if it used alone. For example, if beauvericin, or a derivative, analog or prodrug, or a pharmacologically active salt thereof is administered in combination with a chemotherapeutic agent to target cancer cells, the chemotherapeutic agent can be used at lower dosage or for a shorter duration than if the chemotherapeutic agent is administered without beauvericin, or a derivative, analog or prodrug, or a pharmacologically active salt thereof.

1. Exportin-7

The Examples below show that beauvericin inhibits the reconstitution of isoform A of the progesterone receptor (PR_{A}), a well-established physiological client of Hsp90, with comparable efficacy to the classical Hsp90 inhibitor 17-AAG. Analysis of protein complexes shows that beauvericin decreases the level of Hsp90 in PR_{A} complexes and causes the recruitment of Exportin-7 into these complexes (FIG. 1C). Exportin-7 is thought to be a general nuclear export factor (Mingot, et al., EMBO J, 23, 3227-3236 (2004) but it has not been linked to PR or Hsp90 signaling pathways, however, it is believed that its presence is important for the inhibitory activity of beauvericin.

Therefore, Exportin-7 expression or availability can be used to modulate the Hsp90 inhibitor activity of beauvericin. For example, in some embodiments, an agent is administered to the subject to increase expression or availability of Exportin-7. The agent can be a small molecule, nucleic acid, protein, or any other agent effective for increasing the expression or availability of Exportin-7.

The agent can be an agent that increases expression of endogenous Exportin-7. In one embodiment, the agent is an Exportin-7 transcription factor.

The agent can be purified or recombinant Exportin-7, for a fusion protein thereof. In some embodiments, the agent is recombinant Exportin-7, a nucleic acid encoding Exportin-7, or an expression vector including a nucleic acid encoding Exportin-7 operably linked to expression control sequences needed for expression of recombinant Exportin-7 in a subject. In some embodiments, the agent is an Exportin-7 fusion protein including Exportin-7, or a functional fragment thereof, and protein transduction domain that increases delivery of the Exportin-7 into the interior of the cells. The fusion protein can alternatively or additionally include a cell targeting signal, a localization signal, or a combination thereof.

Nucleic acid and amino acid sequences for Exportin-7 are known in the art. See, for example, UniProtKB accession no. Q9UIA9 (XPO7_HUMAN) which is specifically incorporated by referenced herein in its entirety. Therefore, Exportin-7 can have the amino acid sequence

MADHVQSLAQ LENLCKQLYE TTDTTTRLQA EKALVEFTNS
PDCLSKCQLL LERGSSSYSQ LLAATCLTKL VSRTNNPLPL
EQRIDIRNYV LNYLATRPKL ATFVTQALIQ LYARITKLGW
FDCQKDDYVF RNAITDVTRF LQDSVEYCII GVTILSQLTN
EINQADTTHP LTKHRKIASS FRDSSLFDIF TLSCNLLKQA
SGKNLNLNDE SQHGLLMQLL KLTHNCLNFD FIGTSTDESS
DDLCTVQIPT SWRSAFLDSS TLQLFFDLYH SIPPSFSPLV
LSCLVQIASV RRSLFNNAER AKFLSHLVDG VKRILENPQS
LSDPNNYHEF CRLLARLKSN YQLGELVKVE NYPEVIRLIA
NFTVTSLQHW EFAPNSVHYL LSLWQRLAAS VPYVKATEPH
MLETYTPEVT KAYITSRLES VHIILRDGLE DPLEDTGLVQ
QQLDQLSTIG RCEYEKTCAL LVQLFDQSAQ SYQELLQSAS
ASPMDIAVQE GRLTWLVYII GAVIGGRVSF ASTDEQDAMD
GELVCRVLQL MNLTDSRLAQ AGNEKLELAM LSFFEQFRKI
YIGDQVQKSS KLYRRLSEVL GLNDETMVLS VFIGKIITNL
KYWGRCEPIT SKTLQLLNDL SIGYSSVRKL VKLSAVQFML
NNHTSEHFSF LGINNQSNLT DMRCRTTFYT ALGRLLMVDL
GEDEDQYEQF MLPLTAAFEA VAQMFSTNSF NEQEAKRTLV
GLVRDLRGIA FAFNAKTSFM MLFEWIYPSY MPILQRAIEL
WYHDPACTTP VLKLMAELVH NRSQRLQFDV SSPNGILLFR
ETSKMITMYG NRILTLGEVP KDQVYALKLK GISICFSMLK
AALSGSYVNF GVFRLYGDDA LDNALQTFIK LLLSIPHSDL
LDYPKLSQSY YSLLEVLTQD HMNFIASLEP HVIMYILSSI
SEGLTALDTM VCTGCCSCLD HIVTYLFKQL SRSTKKRTTP
LNQESDRFLH IMQQHPEMIQ QMLSTVLNII IFEDCRNQWS
MSRPLLGLIL LNEKYFSDLR NSIVNSQPPE KQQAMHLCFE
NLMEGIERNL LTKNRDRFTQ NLSAFRREVN DSMKNSTYGV
NSNDMMS

(SEQ ID NO:1), or a functional fragment, variant, or fusion protein thereof, with 50, 60, 70, 80, 90, 95, 99% sequence identity to SEQ ID NO:1.

In some embodiments, an agent is administered to reduce the expression or activity of Exportin-7. Such agents can be inhibitory nucleic acids designed to target Exportin-7 mRNA, or protein or small molecule inhibitors of Exportin-7.

2. Additional Hsp90 Inhibitors

In some embodiments, the additional therapeutic agents are inhibitors of Hsp90. Other Hsp90 inhibitors are known in the art and include first-generation inhibitors derived from the natural product geldanamycin, such as 17-AAG, 17-DMAG, Retaspimycin HCl (IPI-504), C-11, STA9090, SNX-2112, SNX-5542, NVP-AUY922, CCT018159, VER-49009, BIIB021, Derrubone, Gedunin, Celastrol (tripterine), (−)-epigallocatechin-3-gallate((−)-EGCG), Novobiocin, Radamide, Radicicol, Radicicol oxmie derivates, AT13387, Debio0932, PochoninA-F, or combinations thereof (Hao, et al. Oncology Reports, 23:1483-92 (2010)). A preferred additional therapeutic agent is an Hsp90 inhibitor that binds the N-terminus of Hsp90.

3. Chemotherapeutic Agents

Additional therapeutic agents can also include conventional cancer therapeutics such as chemotherapeutic agents, cytokines, chemokines, and radiation therapy. The majority of chemotherapeutic drugs can be divided in to: alkylating agents, antimetabolites, anthracyclines, plant alkaloids, topoisomerase inhibitors, and other antitumor agents. All of these drugs affect cell division or DNA synthesis and function in some way. Additional therapeutics include monoclonal antibodies and the new tyrosine kinase inhibitors e.g., imatinib mesylate (GLEEVEC® or GLIVEC®), which directly targets a molecular abnormality in certain types of cancer (chronic myelogenous leukemia, gastrointestinal stromal tumors).

In a preferred embodiment the additional therapeutic agent is a chemotherapeutic agent. Representative chemotherapeutic agents include, but are not limited to cisplatin, carboplatin, oxaliplatin, mechlorethamine, cyclophosphamide, chlorambucil, vincristine, vinblastine, vinorelbine, vindesine, taxol and derivatives thereof, irinotecan, topotecan, amsacrine, etoposide, etoposide phosphate, teniposide, epipodophyllotoxins, trastuzumab (HERCEPTIN®), cetuximab, and rituximab (RITUXAN® or MABTHERA®), bevacizumab (AVASTIN®), and combinations thereof.

4. Neurodegenerative Disease Treatments

The additional therapeutic agents can be conventional therapeutic agents for treating a proteinopathy, amyloidosis, or a tauopathy. The additional agents can be determined based on the disease to be treated. For example, if the disease is Alzheimer's disease, the compositions disclosed herein can be co-administered with a conventional Alzheimer's disease treatment such as Aβ42 immunization (Wisniewski and Konietzko, Lancet Neurol., 7:805-811 (2008)), tarenflurbil (Flurizan™, Myriad Pharmaceuticals) which is believed to act by decreasing the production of Aβ42 (Aisen, Lancet Neurol., 7:468-469 (2008)), and tramiprosate (Alzhemed™, Neurochem Inc.) which was designed to bind to beta amyloid peptide and prevent it from reacting with glycosaminoglycans (Aisen et al., Curr. Alzheimer Res., 4:473-478 (2007)).

5. Drugs to Treat Infection

In some embodiments the additional therapeutic agents are agents that treat infection, such as antibacterial and antifungal drugs. Hsp90 enables the emergence and maintenance of fungal drug resistance by mediating resistance to azoles, which inhibit ergosterol biosynthesis and are the most widely deployed antifungals in the clinic, and to echinocandins, which inhibit beta(1, 3)-glucan synthesis and are the only new class of antifungals to reach the clinic in decades. (Cowen et al, Proc Natl Acad Sci USA, 106:2813-23 (2009)). Combination therapy with Hsp90 inhibitors provides means for improving treatment of fungal disease because it prevents the emergence of drug resistance. In one embodiment the additional therapeutic agent is an azole. Representative azoles include, but are not limited to Clotrimazole, Posaconazole, Ravuconazole, Econazole, Ketoconazole, Voriconazole, Fluconazole, Itraconazole, Tebuconazole and Propiconazole. In another embodiment the additional therapeutic agent is an echinocandin. Representative echinocandins include, but are not limited to pneumocandins, Echinocandin B, Cilofungin, Caspofungin, Micafungin (FK463) and Anidulafungin (VER-002, V-echinocandin, LY303366).

6. Other Active Agents

Other active agents that can be used alone, or in combination with beauvericin include, but are not limited to, vitamin supplements, appetite-stimulating medications, medications that help food move through the intestine, nutritional supplements, anti-anxiety medication, anti-depression medication, anti-coagulants, clotting factors, antiemetic medications, antidiarrheal medications, anti-inflammatories, steroids such as corticosteroids or drugs that mimic progesterone, omega-3 fatty acids supplements, eicosapentaenoic acid supplements, anti-inflammatories, anabolic agents, psycho-stimulants, selective androgen-receptor modulators, anti-depressant medications, anti-anxiety medications and analgesics.

### EXAMPLES

### Example 1

### Identification of Small Molecule Inhibitors of Hsp90

### Materials and Methods

Progesterone Receptor (PR) Reconstitution Assay

To identify novel small molecule inhibitors of molecular chaperones, a high-throughput functional screen was developed based on the isoform A of progesterone receptor (PR_{A}), a well-established physiological client of Hsp90, and using rabbit reticulocyte lysate (RRL) as a source of molecular chaperones. This comprehensive functional assay measured the recovery of hormone binding activity of PR_{A} after mild heat treatment. Purified PR was adsorbed onto PR22 antibody bound to protein A that was absorbed on 96 well plates. 100 μl of RRL lysate and ATP regeneration system was added to each well. After incubation for 30 min at 30° C., 0.1 μM [³H]-progesterone (American Radiolabeled Chemicals, Inc #ART 0063) was added. Plates were incubated on ice for 2 h at 4° C. Complexes were then washed three times with 200 μl of reaction buffer (20 mM Tris/HCl, pH 7.5, 5 mM MgCl₂, 2 mM DTT, 0.01% NP-40, 50 mM KCl and 5 mM ATP) and assessed for bound progesterone by liquid scintillation using PerkinElmer Microbeta plate reader.

### Results

Approximately seventy natural products of different chemical scaffolds isolated from Moroccan medicinal plants and their endophytes, as well as other sources were screened. Five novel compounds were identified as potent inhibitors of the Hsp90 chaperone pathway in vitro and in cells (FIG. 1).

### Example 2

### Beauvericin Inhibits Reconstitution of PR_{A}

### Materials and Methods

Beauvericin was added to the PR reconstitution assay as in example 1, and hormone binding activity was measured.

### Results

Compound AD05, beauvericin, was shown to interfere with the activity of the Hsp90 chaperone, as determined by a hormone receptor binding assay. Beauvericin inhibited reconstitution of PR_{A} in RRL with comparable efficacy to the classical Hsp90 inhibitor 17-AAG (FIG. 1). Beauvericin inhibited the ability of the Hsp90 chaperone to refold PR_{A} in a concentration-dependent manner (FIG. 2).

### Example 3

### Inhibition of Hsp90 by Beauvericin Involves Exportin-7

### Materials and Methods

Progesterone Receptor (PR) Reconstitution Assay in Tubes

Purified PR was adsorbed onto PR22 antibody-protein A-sepharose resin beads and was assembled into complexes as described previously (Kosano, et al., J Biol Chem, 273:32973-9 (1998)). Briefly, about 0.05 μM PR was incubated with 100 μl of RRL. After incubation for 30 min at 30° C., 0.1 μM [³H]-progesterone (American Radiolabeled Chemicals, Inc #ART 0063) was added. Samples were incubated on ice for 3 h at 4° C. Complexes were then washed three times with 1 ml reaction buffer (20 mM Tris/HCl, pH 7.5, 5 mM MgCl₂, 2 mM DTT, 0.01% NP-40, 50 mM KCl and 5 mM ATP) and assessed for bound progesterone by liquid scintillation using PerkinElmer Microbeta plate reader, and for composition of protein complexes by SDS-PAGE (10% gel) and Coomassie blue staining.

### Results

Beauvericin decreased the level of Hsp90 in PR_{A} complexes and caused the recruitment of a novel protein into these complexes, as determined by SDS-PAGE analysis of the complexes formed by purified proteins (FIG. 3). The novel protein was identified by mass spectrometry analysis as Exportin-7. Exportin-7 is thought to be a general nuclear export factor (Mingot, et al., EMBO J, 23, 3227-3236 (2004)) but it has not been linked to PR or Hsp90 signaling pathways. These results indicated Exportin-7 is involved in the mechanism of chaperone inhibition by beauvericin.

### Example 4

### Mechanism of Hsp90 Inhibition by Beauvericin is Different from that of the Classical Inhibitor 17-AAG

### Materials and Methods

Progesterone Receptor (PR) Reconstitution Assay Using the Five Purified Chaperones.

Purified PR was adsorbed onto PR22 antibody-protein A-sepharose resin beads and was assembled into complexes as described previously (Kosano, et al., J Biol Chem 273:32973-9 (1998)). Briefly, about 0.05 μM PR was incubated with 1.4 μM Hsp70, 0.8 μM Hsp90β, 0.2 μM Hsp40 (Ydj), 0.08 μM HOP and 2.6 μM p23 in reaction buffer (20 mM Tris/HCl, pH 7.5, 5 mM MgCl₂, 2 mM DTT, 0.01% NP-40, 50 mM KCl and 5 mM ATP). After incubation for 30 min at 30° C., 0.1 μM [³H]-progesterone (American Radiolabeled Chemicals, Inc #ART 0063) was added. Samples were incubated on ice for 3 h at 4° C. Complexes were then washed three times with 1 ml of reaction buffer and assessed for bound progesterone by liquid scintillation using PerkinElmer Microbeta plate reader and for composition of protein complexes by SDS-PAGE (10% gel) and Coomassie blue staining.

### Results

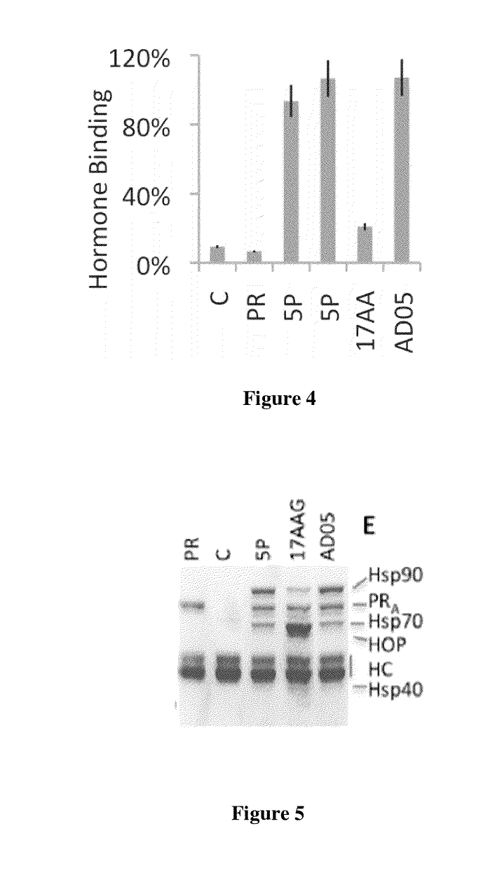

The molecular underpinnings and mechanism of inhibition by beauvericin was further assessed by the PR_{A} reconstitution assay using purified forms of the five well-characterized chaperone proteins Hsp90, Hsp70, Hsp40, HOP and p23 (FIG. 4). The complexes formed during the assay were subsequently analyzed y SDS-PAGE analysis (FIG. 5). Surprisingly, beauvericin had no inhibitory activity on this five-protein system, indicating that the inhibition of chaperone activity by beauvericin was dependent upon the presence of Exportin-7, and maybe other unknown factors. These data indicate that beauvericin inhibits the Hsp90 chaperone pathway through a mechanism distinct from that of 17-AAG.

### Example 5

### Beauvericin has Cytotoxic Activity in Cancer Cells

### Materials and Methods

Breast cancer cell lines Hs578T and MDA-MB-453 at 40% confluence in 6-well plates (Corning #3516) were cultured for 24 h and then treated with various concentrations of beauvericin. Cells were harvested at indicated time and cell lysates were made. 15 μg of protein lysate were analyzed by Western blotting using specific antibodies for the indicated proteins.

### Results

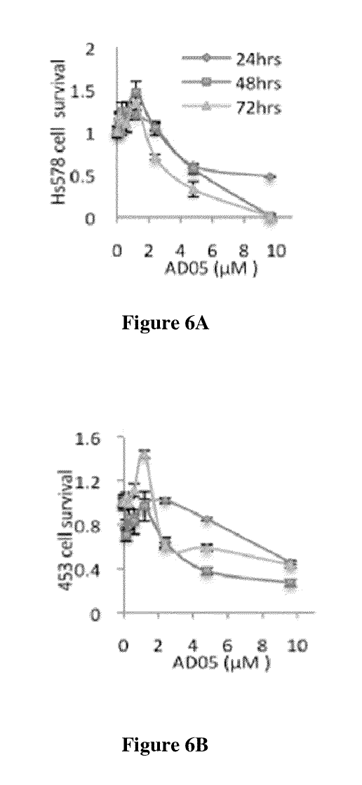

A cytotoxicity assay using the two breast cancer cell lines Hs578T and MDA-MB-453 was utilized to evaluate the in vitro antitumor efficacy of beauvericin (FIGS. 6A and 6B). At the cellular level, beauvericin showed potent in vitro cytotoxicity to both breast cancer cell lines Hs578T (FIG. 6A) and MDA-MB-453 (FIG. 6B). Survival of cell lines in the presence of beauvericin correlated negatively with both dose and exposure time.

### Example 6

### Beauvericin does not Induce Cellular Heat Shock Responses

### Materials and Methods

Cell lysates prepared in Example 5 were analyzed for markers for heat shock response using western blot. These include overexpression of Hsp70, Hsp27, Hsp40 and HOP.

### Results

Evaluation of the Hsp90 inactivation molecular signature by Western blot analysis of chaperone proteins following beauvericin treatment clearly established that beauvericin inhibited the Hsp90 chaperone in both the Hs578T (FIG. 7A) and MDA-MB-453 (FIG. 7B) cancer cell lines. Indeed, treatment with beauvericin caused cellular degradation of several kinase protein clients of Hsp90 (AKT, pAKT and CDK4, ILK, Her2 and Her3) and the glucocorticoid receptor (GR) (FIGS. 7A-B). However, there was variation in how beauvericin affected the different cancer cell lines. For instance, AKT was destabilized in Hs578T cells but not in MDA-MB-453 cells and the reverse profile is observed for Her2.

In contrast to the known Hsp90 N-terminus inhibitor 17-AAG, beauvericin does not induce overexpression of Hsp70 and Hsp27. Furthermore, beauvericin down-regulates Hsp40 and does not induce overexpression of HOP (FIG. 7). Together these data indicate that beauvericin does not induce cellular heat shock response. They also further confirm that beauvericin inhibits the Hsp90 chaperone through a novel mechanism distinct from that of 17-AAG.

### Example 7

### Beauvericin Interferes with Nucleocytoplasmic Trafficking

### Materials and Methods

Immunocytochemistry and Fluorescence Microscopy.

HeLa-PR_{B} cells were grown in 24-well plates (Corning #3337) on micro-cover glasses (Electron Microscopy Sciences) to about 50% confluence in MEM, 1× (Cellgro #10-010-CV) medium supplemented with 10% fetal bovine serum. Cells were treated with 3 μM beauvericin (or DMSO control) for 24 h. Cells were fixed with 0.1 M PIPES, pH 6.95, 1 mM EGTA, pH 8.0, 3 mM MgSO₄, 3% paraformaldehyde, permeabilized with 0.1% triton X-100, and blocked with 10% fetal bovine serum with 5% glycerol and stored at 4° C. Primary antibodies against PR_{B}, GR and Hsp90 and secondary antibodies were prepared in the blocking buffer. Coverslips were washed and mounted on slides with ProLong Gold anti-fade reagent with 4′,6-diamidino-2-phenylindole (Invitrogen). Cells were imaged using a Zeiss Imager M1 microscope. Deconvolution of Z-stack images was done using an inverse filter algorithm with auto-linear normalization.

### Results

Because beauvericin treatment induces incorporation of Exportin-7 into PR_{A} complexes, whether beauvericin affects the nucleocytoplasmic trafficking of steroid receptors was tested using HeLa cells stably expressing the isoform B of PR (PR_{B}). Surprisingly, beauvericin treatment reproducibly blocked the PR_{B} recognition by the monoclonal antibody PR6 in immunocytochemistry (data not shown). However, Western blot analysis demonstrated that PR_{B} is not destabilized in these cells. On the other hand, labeling of glucocorticoid receptor (GR) indicated that beauvericin clearly affected GR localization and seemed to lock it in the nucleus (FIGS. 8A-B, lower panels).

We claim:

1. A method of inhibiting the Hsp90 chaperone pathway comprising contacting one or more cells expressing Hsp90 with an effective amount of a beauvericin, a derivative, analog, prodrug, or a pharmacologically active salt thereof to reduce, decrease, or inhibit the Hsp90 chaperone pathway in the cells compared to a control.

2. The method of claim 1 wherein the beauvericin, synthetic beauvericin, or derivative, analog or prodrug, or pharmacologically active salt thereof inhibits formation of, or increases degradation of Hsp90 complexes.

3. The method of claim 2 wherein the Hsp90 complex includes one or more client proteins selected from the group consisting of AKT, pAKT and CDK4, ILK, Her2, Her3 and the glucocorticoid receptor (GR).

4. The method of claim 1 wherein the beauvericin reduces or inhibits Hsp90-mediated folding, activation, assembly, or function of proteins.

5. The method of claim 1 wherein the cells are under stress or transforming pressure.

6. The method of claim 1 wherein the cells are diseased or pathogenic.

7. The method of claim 1 wherein the naturally occurring beauvericin, synthetic beauvericin, or derivative, analog or prodrug, or pharmacologically active salt thereof reduces the viability of the contacted cells.

8. The method of claim 7 wherein the naturally occurring beauvericin, synthetic beauvericin, or derivative, analog or prodrug, or pharmacologically active salt thereof increases apoptosis of the cells.

9. The method of claim 1 wherein the naturally occurring beauvericin, synthetic beauvericin, or derivative, analog or prodrug, or pharmacologically active salt thereof does not increase expression of Hsp70, Hsp24, Hsp40, or HOP.

10. The method of claim 1 wherein the contacting occurs in vivo in a subject in need thereof of.

11. The method claim 10 wherein in the subject has a disease or disorder selected from the group consisting of cancer, an inflammatory disease or disorder, a neurodegenerative disease, or an infectious disease.

12. The method of claim 11 further comprising administering to the subject one or more additional therapeutic agents.

13. The method of claim 12 wherein the second therapeutic agent is an agent that increases expression or availability of Exportin-7, or the incorporation of Exportin 7 into Hsp90 client protein complexes.

14. A method of treating cancer comprising administering to a subject with cancer an effective amount of a naturally occurring beauvericin, synthetic beauvericin, or derivative, analog or prodrug, or pharmacologically active salt thereof to reduce or inhibit one or more symptoms of the cancer.

15. The method of claim 12 wherein the cancer is selected from the group consisting of lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

16. A pharmaceutical composition comprising an effective amount of a naturally occurring beauvericin, a synthetic beauvericin, or a derivative, analog or prodrug, or a pharmacologically active salt thereof to reduce, decrease, to inhibit the Hsp90 chaperone pathway in cells of a subject compared to a control, and a pharmaceutically acceptable carrier.

17. The pharmaceutical composition of claim 17 further comprising one or more additional therapeutic agents.
